(19) United States
(12) Patent Application Publication
(10) Pub. No.: US 2006/0228307 A1
(43) Pub. Date: Oct. 12, 2006

(54) Structures and methods for delivering topical compositions

(75) Inventor: Robert K. Larsen, Sandy, UT (US)

Correspondence Address: Parsons Behle & Latimer;Suite 1800, 201 South Main Street, P. O. Box 45898, Salt Lake City, UT, 84111, US

(73) Assignee: Cao Group, Inc.

(21) Appl. No.: 11/397,300

(22) Filed: Apr. 4, 2006

Related U.S. Application Data
(60) Provisional application No. 60/669,684, filed on Apr. 8, 2005.

(51) Int. Cl. A61K 8/22; A61K 8/44

(52) U.S. Cl. 424/53

(57) ABSTRACT

Structures and methods for delivering topical compositions are disclosed that fully integrate the topical composition with a carrier. One example adds a liquid topical composition, such as a dental bleach, to a liquid polymer carrier, mixes the two, and then solidifies the mixture. The solidified mixture is a delivery device which is then contacted with the human body in order for the topical composition have its desired effect on the body. Another example embeds the topical composition in an absorbent material. When the absorbent material is contacted with a portion of the human body, the topical composition can have its desired effect.

### PRIORITY

Priority is claimed to U.S. Provisional Patent Application Ser. No. 60/669,684 filed on Apr. 8, 2005.

### BACKGROUND

In the prior art, an effort was made to apply topical compositions to areas of the human body, including the teeth, by placing the topical composition on a plastic substrate and then adhering it to the human body for treatment. In such instances, the prior art and substrate were two separate layers which were neither chemically nor physically integrated.

### SUMMARY

Structures and methods for delivering topical compositions are disclosed that fully integrate the topical composition with a carrier. One example adds a liquid topical composition, such as a dental bleach, to a liquid polymer carrier, mixes the two, and then solidifies the mixture. The solidified mixture is a delivery device which is then contacted with the human body in order for the topical composition have its desired effect on the body. Another example embeds the topical composition in an absorbent material. When the absorbent material is contacted with a portion of the human body, the topical composition can have its desired effect.

### BRIEF DESCRIPTION OF DRAWINGS

FIG. 1 depicts a perspective view of a topical delivery strip.

FIG. 2 depicts a side view of a topical delivery strip.

FIG. 3 depicts a topical delivery strip applied to a tooth.

### DETAILED DESCRIPTION

The present disclosure relates to a structures and methods for delivering chemical agents in a topical manner by homogeneously incorporating or embedding said chemical agents into the application device, or carrier. Said chemical agents may be incorporated or embedded into various application devices or carriers, such as an applications device having a composition containing high molecular weight polymer materials in concentrations of 0.01% to 99.9% by weight. “High molecular weight polymer materials” is defined to mean polymer materials having a weight average molecular weight of at least about 1,000 determined according to the gel permeation chromatography method. Suitable polymer materials includes polyvinyl alcohol (PVA) and its partially hydrolyzed polyvinyl acetate equivalents, polyvinyl acetate (PVAc), polyvinylpyrrilidone (PVP), polyethylene glycol (also referred to as poly oxyethylene), polypropylene glycol, polyacrylic acid and its derivatives, carboxymethyl cellulose and related cellulosic compounds, and xantham gum and related resin gums. Other carriers may be employed with a composition of silicas, silicates, and silanes. Another alternative is to embed the chemical agents into a paper, cloth or other absorbent material as a carrier that will later release the chemical agents when desired.

An example structure and method of incorporation or embedding is to prepare a polymer composition in a liquid state, to which is added the chemical agent of choice. The polymer composition incorporating the chemical agent is then converted to a solid state to form the application device. The application device is then applied topically to a number of structures of the human body for the purpose of releasing the chemical agent onto or into the structure of the body. This represents a substantially new technology over current technologies in that the chemical agent to be delivered is not applied as a separate composition to the polymer delivery device, but is homogeneously dispersed or embedded within the polymer delivery device, creating a delivery system that is physically one composition. Said polymer device (carrier) may be used independently, or combined with an additional laminate backing. Said polymer device may be used independently or in conjunction with other topical application systems or as a dual or multi-component system.

Another example does not rely on chemical incorporation of the chemical agent into the carrier, but uses a carrier of absorbent material such as paper or cloth. The absorbency of the carrier will tend to pick up and hold the chemical agent for later leaching out into a desired surface of the human body to carry out a treatment. The carrier may be super-saturated with the chemical agent such that the chemical agent readily flows or leaches out of it, or it may be only fully or partially saturated in order to provide a slow release of the chemical agent to a human subject over time.

One example carrier uses polyvinyl alcohol (PVA) as at least one of the polymer materials for the delivery device. Two or more polymers may be mixed or associated to form a carrier. Another example uses an absorbent or hyrophillic paper to absorb, hold and later release the chemical agent. Another example uses an absorbent or hydrophillic cloth to absorb, hold and later release the chemical agent.

The carrier may be used to store and later release many desired chemical agents for many different purposes. One example is the delivery and the use of peroxides such as hydrogen peroxide, carbamide peroxide, and/or sodium peroxide as the chemical agent, to be delivered to tooth structures for the purpose of whitening teeth. Another example is delivery of lidocaine, benzocaine, and/or other anesthetic pharmaceuticals as the chemical agent, for the purpose of delivering said anesthetics to injured or irritated structures of the body for the purpose of reducing pain in said affected structures. Another example uses bacitracin and other antibacterial and/or antimicrobial pharmaceuticals as the chemical agent, for the purpose of delivering said other antibacterial and/or antimicrobial pharmaceuticals to injured or irritated structures of the body for the purpose of preventing or eliminating infection by foreign materials in said affected structures.

### Example 1

A delivery device for the purpose of whitening teeth is prepared. A polymer delivery material of the following composition can be prepared:

| Component | % by weight |
|---|---|
| Water | 0.01-95.00 |
| PVA | 0.01-95.00 |
| Glycerin | 0.01-50.00 |

These components are processed to form a homogenous liquid composition, to which can be added hydrogen peroxide in concentrations of 0.01% to 40.00% by weight of the total composition. The combined composition is mixed to create a homogeneous mixture. The mixture is then deposited onto a large flat surface, which can incorporate a release backing. The mixture is spread out across the flat surface to form a thin layer with a minimum thickness of 1 micrometer. The mixture is then allowed to remain exposed to air until the mixture changes to a solid form. At this point the solid film that is formed may be released from the flat surface, or subdivided into single-application segments.

A composition as outlined was prepared using water of a concentration of 56% by weight, PVA of a concentration of 12% by weight, glycerin of a concentration of 10% by weight, and a 50% by weight solution of hydrogen peroxide in water of a concentration of 22% by weight. The mixture was then processed as outlined above to create the desired device.

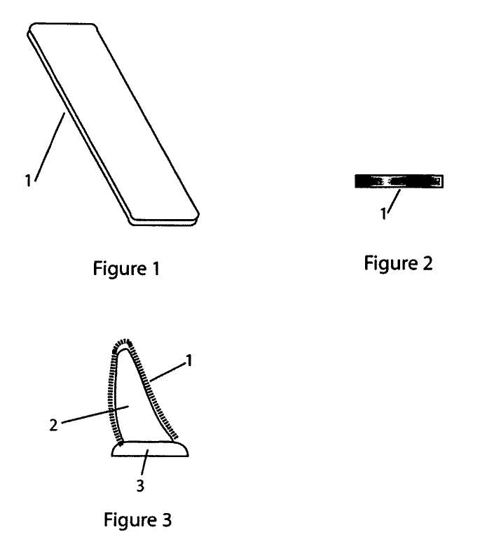

FIG. 1 is a perspective view of a strip in accordance with principles herein in a possible geometric shape 1 as would be suitable for topical application to the human body.

FIG. 2 is a perspective view of a strip in accordance with the principles herein 1 illustrating example desired thickness of the material, said thickness indicated above as an example minimum of 1 micrometer.

FIG. 3 is an illustration of the application of a completed strip 1 as prepared according to Example 1 and applied to the tooth structure 2. The geometric shape of 1 can be adjusted such that 1 does not come in contact with gum tissue 3 at the base of tooth structure 2. It is envisioned that the geometric shape of the strip 1 could be such that an individual tooth 2 or an array of several teeth can be treated simultaneously by the application of the strip 1.

While the present invention has been described and illustrated in conjunction with a number of specific embodiments, those skilled in the art will appreciate that variations and modifications may be made without departing from the principles of the invention as herein illustrated, described, and claimed. The present invention may be embodied in other specific forms without departing from its spirit or essential characteristics. The described embodiments are to be considered in all respects as only illustrative, and not restrictive. All changes which come within the meaning and range of equivalency of the claims are to be embraced within their scope.

I claim:

1. A method for making and using a topical delivery strip comprising:
selecting a topical composition for delivery to a portion of a human body,
selecting a first polymer,
creating a solution of said topical composition and said first polymer,
permitting said solution to solidify,
forming said solution into a strip of desired size and shape,
applying said strip to a portion of a human body which may benefit from application of said topical composition, and
permitting said composition to be released from said strip into said portion of a human body.

2. A method as recited in claim 1 herein said polymer has a weight average molecular weight of at least 1000 as determined by gel permeation chromoatography.

3. A method as recited in claim 1 wherein said polymer is selected from the group consisting of PVA, PVAc, PVP, polyethylene glycol, polypropylene glycol, polyacrylic acid, carboxymethyl cellulose, xantham gum and resin gum.

4. A method as recited in claim 1 further comprising the steps of:
selecting a second polymer, and
mixing said second polymer in said composition.

5. A method as recited in claim 1 wherein said polymer serves as a carrier for said topical composition.

6. A method as recited in claim 1 wherein said topical composition includes a peroxide.

7. A method as recited in claim 6 wherein said peroxide is selected from the group consisting of carbamide peroxide, hydrogen peroxide and sodium peroxide.

8. A method as recited in claim 7 wherein said topical composition serves to bleach teeth.

9. A method as recited in claim 1 wherein said topical composition includes an anesthetic.

10. A method as recited in claim 9 wherein said anesthetic is selected from the group consisting of lidocaine and benzocaine.

11. A method as recited in claim 1 wherein said topical composition is selected from the group consisting of antibacterials and antimicrobials.

12. A method as recited in claim 1 further comprising the step of spreading said solution over a flat surface and wherein said step of permitting said solution to solidify causes said solution to form a sheet.

13. A method as recited in claim 8 wherein said peroxide is present in said solution in the range of from about 0.01% to about 40.0$ by weight of total composition.

14. A method for making and using a topical delivery strip comprising:
selecting a topical composition for delivery to a portion of a human body,
selecting a carrier,
creating a solution of said topical composition and said carrier,
permitting said solution to solidify,
forming said solution into a strip of desired size and shape,
applying said strip to a portion of a human body which may benefit from application of said topical composition, and
permitting said composition to be released from said strip into said portion of a human body.

15. A method as recited in claim 14 wherein said carrier is selected from the group consisting of silicas, silicates, and silanes.

16. A method as recited in claim 1 wherein said carrier is selected from the group consisting of PVA, PVAc, PVP, polyethylene glycol, polypropylene glycol, polyacrylic acid, carboxymethyl cellulose, xantham gum and resin gum.

17. A method for making and using a topical delivery strip comprising:
selecting a topical composition for delivery to a portion of a human body,
selecting an absorbent material to serve as a carrier,
associating said topical composition with said absorbent material.
forming said absorbent material into a delivery strip,
applying said strip to a portion of a human body which may benefit from
application of said topical composition, and
permitting said composition to be released from said strip into said portion of a human body.

18. A method as recited in claim 17 wherein said absorbent material is selected from the group consisting of cloth and paper.

19. A method as recited in claim 17 wherein said absorbent material is super-saturated with said topical composition.

20. A method as recited in claim 17 wherein said topical composition is a dental bleach.
